(19) United States
(12) Patent Application Publication
(10) Pub. No.: US 2025/0210189 A1
(43) Pub. Date: Jun. 26, 2025

(54) MEASUREMENT DEVICE, CONTROL METHOD, AND CONTROL RECORDING MEDIUM

(71) Applicant: OMRON HEALTHCARE Co., Ltd., Kyoto (JP)

(72) Inventors: Nozomi NAKAWAKI, Kyoto (JP); Nobuo KUBO, Kyoto (JP); Yasunori FUKUDA, Kyoto (JP); Kazuki KUMAGAI, Kyoto (JP); Yuya HIGUCHI, Kyoto (JP)

(21) Appl. No.: 19/075,481

(22) Filed: Mar. 10, 2025

Related U.S. Application Data
(63) Continuation of application No. PCT/JP2023/040264, filed on Nov. 8, 2023.

(30) Foreign Application Priority Data
Mar. 8, 2023 (JP) 2023-035939

(51) Int. Cl. G16H 40/63

(52) U.S. Cl. CPC G16H 40/63

(57) ABSTRACT

A measurement device, a control method, and a control recording medium that can improve usability in measurement of biological data. A measurement device according to one aspect of the present invention includes a control unit that performs measurement using a sensor that acquires biological data, and a nonvolatile memory. Then, the control unit starts measurement and data erasure of the nonvolatile memory upon receiving an instruction for measurement, and writes, into the nonvolatile memory, biological data obtained in the measurement upon completing the data erasure.

### CROSS-REFERENCE TO RELATED APPLICATIONS

This application is the U.S. national stage application filed pursuant to 35 U.S.C. 365(c) and 120 as a continuation of International Patent Application No. PCT/JP2023/040264, filed Nov. 8, 2023, which application claims priority to Japanese Patent Application No. 2023-035939, filed Mar. 8, 2023, which applications are incorporated herein by reference in their entireties.

### TECHNICAL FIELD

The present invention relates to a measurement device, a control method, and a control recording medium.

### BACKGROUND ART

There is a known health device (such as a sphygmomanometer) equipped with a Bluetooth (registered trademark) Low Energy (BLE) communication function. This health device can transmit measurement data to, for example, an external device by BLE communication, and acquire and manage the measurement data of the health device by the external device. In this case, the health device saves the measurement data in, for example, a memory in the health device, and collectively transmits the saved data to the external device. When the health device saves the measurement data, the health device may save the measurement result after completion of the measurement of the data, or may sequentially save data while measuring the data when the data is large in capacity. In a case of saving data large in capacity, for example, a flash memory having a large capacity and being relatively inexpensive may be used.

For example, there is a known sphygmomanometer that can store a pulse wave detected by a blood pressure calculation means and a blood pressure value calculated from the pulse wave into a storage means in association with measurement date and time, output the stored data to the outside from an output terminal, and display a signal level of the pulse wave in a time-series graph (Patent Document 1).

### CITATION LIST

### Patent Literature

Patent Document 1: JP 2007-098003 A

### SUMMARY

### Technical Problem

However, in a case of use of the flash memory, for example, when the measurement data is written into the memory, erasure processing of the memory data is necessary, and many flash memories have specifications for erasing data in collective units such as sector units. Therefore, there is a problem of taking time to erase the data.

Methods of erasing memory data at the time of data measurement include, for example, a method of erasing the memory data first when a measurement start operation is performed and starting the measurement of the data after the erasure. However, in this case, if the data size to be erased is large, it takes time to erase, and the start of data measurement is delayed.

Methods of erasing memory data include, for example, a method of erasing a memory area for one time of measurement after completion of data measurement. However, in this case, although the erasure at the start of the measurement is unnecessary, an erased area needs to be always secured in the memory, and the number of storable data is reduced. In a case where measurement is continuously executed a plurality of times due to a button operation error or the like, the start of the second time is delayed due to the erasure processing, and the usability is deteriorated. Therefore, there is still room for further improvement in the method of erasing or saving, into the memory, memory data when data measured by a health device or the like is saved in the memory.

According to the sphygmomanometer of Patent Document 1, it is possible to clearly indicate what pulse wave the blood pressure calculation means detected and what feature point of the detected pulse wave to use to calculate the blood pressure value, and therefore it is possible for the subject himself/herself to confirm whether the blood pressure value is normally calculated. However, Patent Document 1 does not describe erasure of memory data and saving of memory data into a memory when measured biological information is saved in the memory.

One aspect of the present invention has been made in view of such circumstances, and an object thereof is to provide a measurement device, a control method, and a control recording medium that can improve usability in measurement of biological data.

### Solution to Problem

The present invention employs the following configurations to solve the above-described problems.
(1)

A measurement device including: a control unit that performs measurement using a sensor that acquires biological data; and a memory, in which
the control unit
starts the measurement and data erasure of the memory upon receiving an instruction for the measurement, and
writes biological data obtained in the measurement to the memory upon completing the

According to (1), by executing the measurement based on the biological data in parallel with the data erasure of the memory, it is possible to start the measurement without waiting for completion of the data erasure. Therefore, the time from the measurement instruction to starting of the measurement can be shortened, and the waiting time for the user can be reduced. This can suppress deterioration of usability due to saving processing of large-capacity biological data while saving the large-capacity biological data into the memory.
(2)

The measurement device according to (1), in which
the memory is a flash memory.

According to (2), it is possible to reduce the waiting time for the user to wait until the data erasure of the flash memory is completed.
(3)

The measurement device according to (1) or (2), in which
the biological data is pulse wave data.

As in (3), for example, pulse wave data is preferable as biological data measured by the measurement device. Note that the pulse wave may be a pressure pulse wave obtained by measuring a change in pressure applied to a blood vessel, or may be a volume pulse wave obtained by measuring a change in blood volume in the blood vessel.
(4)

The measurement device according to (3), in which
the control unit outputs a blood pressure measurement result based on the pulse wave data.

According to (4), the blood pressure measurement result can be displayed on the display unit of the measurement device, for example.
(5)

The measurement device according to (3) or (4) including:
a cuff including a band-shaped body attached around a measurement target site and configured to change a compression pressure on the measurement target site, and the sensor disposed between the band-shaped body and the measurement target site, in which the measurement includes a pressurization process of increasing the compression pressure and a depressurization process of decreasing the compression pressure after the pressurization process.

Since the measurement of pulse wave data includes the pressurization process first, and the sensing data during the pressurization process is relatively unimportant, even if the measurement is started while memory erasure is performed and the sensing data at the beginning of the measurement is lost, the influence on the analysis and the like is small. Therefore, as in (5), pulse wave data measured using a cuff is preferable as biological data measured by the measurement device.
(6)

The measurement device according to (5), in which
the control unit does not write, into the memory, biological data obtained in the pressurization process, and writes, into the memory, biological data obtained in the depressurization process.

According to (6), since memory erasure is performed together with the pressurization process, the waiting time for the user at the time of start of the depressurization process can be reduced.
(7)

The measurement device according to (5), in which
the control unit does not write, into the memory, biological data obtained before completion of the data erasure in the pressurization process, and writes, into the memory, biological data obtained after completion of the data erasure in the pressurization process and biological data obtained in the depressurization process.

According to (7), since memory erasure is performed together with the pressurization process, the waiting time for the user at the time of start of the depressurization process can be reduced. Biological data obtained after completion of data erasure in the pressurization process can also be written into the memory.
(8)

The measurement device according to any one of (1) to (5), in which
the control unit
buffers biological data obtained in the measurement until the data erasure is completed, and
writes, into the memory, the biological data buffered upon completing the data erasure.

According to (8), by buffering also the biological data obtained before the data erasure is completed, it is possible to write also this biological data into the memory after the completion of the data erasure.
(9)
(1) The measurement device according to any one of (1) to (8), in which
the control unit transmits, to an information terminal, the biological data written in the memory.

According to (9), the biological data measured by the measurement device can be received by an information terminal such as a smartphone or a cloud server, and the received biological data can be analyzed by the information terminal.
(10)

The measurement device according to (9), in which
the control unit includes
a first processor that performs the measurement, and
a second processor that performs wireless communication with the information terminal,
the memory is connected to the second processor,
the first processor transmits the biological data to the second processor and writes the biological data into the memory, and
the second processor transmits, to the information terminal, the biological data written in the memory.

In the configuration in which measurement based on the biological data is executed in parallel with data erasure of the memory, if these processing are performed by one processor, a processing load increases, and a delay occurs in the processing such as the measurement. Therefore, as in (10), by providing the second processor that performs erasure of the memory, writing to the memory, and wireless communication separately from the first processor that performs measurement, the processing load for erasing the erasure target of the memory, writing the pulse wave data to the memory, and transmitting the pulse wave data to the information terminal can be distributed to the second processor, and the processing load of the first processor can be reduced. This can suppress a delay in processing such as measurement by the first processor.
(11)

The measurement device according to (10), in which
the first processor
specifies an address of a write destination in the memory and causes the second processor to write the biological data into the memory, and
specifies an address of a read source in the memory and causes the second processor to read the biological data from the memory and transmit the biological data to the information terminal.

According to (11), since the first processor is configured to specify the address of the memory connected to the second processor and instruct the second processor to write, read, and transmit the biological data, flow control is unnecessary in an interface (e.g., universal asynchronous receiver transmitter (UART) or the like) between the first processor and the second processor, and the transfer speed of the biological data to the information terminal can be improved. Since the second processor may perform writing of information to a specified address in the memory and reading and transmission of information from the specified address, the second processor can have a simple configuration. By an instruction to the second processor, the first processor can flexibly perform writing of the biological data to the memory, reading of the biological data from the memory, and transmission of the read biological data. However, since it is not necessary for the first processor itself to perform processing with a large load such as write processing, read processing, and transmission processing of biological data and the like, it is possible to reduce the processing load of the first processor as in (7) described above.
(12)

The measurement device according to (10) or (11), in which
the first processor
specifies an address of an erasure target in the memory and causes the second processor to erase the biological data from the memory.

According to (12), since the first processor is configured to specify the address of the memory connected to the second processor and instruct the second processor to erase the erasure target, the flow control is unnecessary in the interface between the first processor and the second processor, and the transfer speed of the biological data to the information terminal can be improved. Since the second processor may erase the erasure target of a specified address in the memory, the second processor can have a simple configuration. By an instruction to the second processor, the first processor can flexibly erase the erasure target in the memory. However, since it is not necessary for the first processor itself to perform processing with a large load such as the erasure processing of the erasure target, it is possible to reduce the processing load of the first processor as in (7) described above.
(13)

The measurement device according to (11) or (12), in which
the memory includes an area allocated for the biological data, and
the address of the write destination and the address of the read source are addresses in the area.

According to (13), by providing the memory with an area in which information other than biological data is not written, it is possible to suppress interference between writing of the biological data by an instruction from the first processor to the second processor and writing of other information by the second processor.
(14)

The measurement device according to any of (10) to (13), in which
the memory is inaccessible from the first processor.

According to (14), as compared with a configuration in which one memory is shared by the first processor and the second processor, access processing can be distributed and sped up.
(15)

A control method for a measurement device including a control unit that performs measurement using a sensor that acquires biological data, and a memory, in which
the control unit
starts the measurement and data erasure of the memory upon receiving an instruction for the measurement, and
writes biological data obtained in the measurement to the memory upon completing the data erasure.

According to (15), by executing the measurement based on the biological data in parallel with the data erasure of the memory, it is possible to start the measurement without waiting for completion of the data erasure. Therefore, the time from the measurement instruction to the starting of the measurement can be shortened, and the waiting time for the user can be reduced. This can suppress deterioration of usability due to saving processing of large-capacity biological data while saving the large-capacity biological data into the memory.
(16)

A control recording medium of a measurement device including a control unit that performs measurement using a sensor that acquires biological data, and a memory, the control recording medium for causing
the control unit to execute processing of
starting the measurement and data erasure of the memory upon receiving an instruction for the measurement, and
writing biological data obtained in the measurement to the memory upon completing the

According to (16), by executing the measurement based on the biological data in parallel with the data erasure of the memory, it is possible to start the measurement without waiting for completion of the data erasure. Therefore, the time from the measurement instruction to the starting of measurement can be shortened, and the waiting time for the user can be reduced. This can suppress deterioration of usability due to saving processing of large-capacity biological data while saving the large-capacity biological data into the memory.

### Advantageous Effects of Invention

According to the present invention, it is possible to provide a measurement device, a control method, and a control recording medium that can improve usability in measurement of biological data.

### BRIEF DESCRIPTION OF DRAWINGS

FIG. 1 is a view illustrating an information management system including a measurement device of the present invention and an information terminal that performs wireless communication with the measurement device.

FIG. 2 is a view illustrating a sphygmomanometer that is an example of the measurement device.

FIG. 3 is a view illustrating an example in which the information terminal is connected to a network.

FIG. 4 is a block diagram illustrating a configuration of the measurement device.

FIG. 5 is a block diagram illustrating a configuration of the information terminal.

FIG. 6 is a sequence diagram illustrating operations of a main MCU, a communication IC, and a nonvolatile memory in the measurement device.

FIG. 7 is a view illustrating an example of biological data to be measured by the measurement device.

FIG. 8 is a view illustrating an example of a first flow of pressure pulse wave data measured by a sphygmomanometer.

FIG. 9 is a view illustrating an example of a second flow of pressure pulse wave data measured by the sphygmomanometer.

FIG. 10 is a view illustrating an example of a third flow of pressure pulse wave data measured by the sphygmomanometer.

FIG. 11 is a view illustrating an example of a fourth flow of pressure pulse wave data measured by the sphygmomanometer.

FIG. 12 is a view illustrating an example of a fifth flow of pressure pulse wave data measured by the sphygmomanometer.

### DESCRIPTION OF EMBODIMENTS

An embodiment according to one aspect of the present invention will be described below based on the drawings.

### § 1 APPLICATION EXAMPLE

### Information Management System 100 Applied with Present Invention

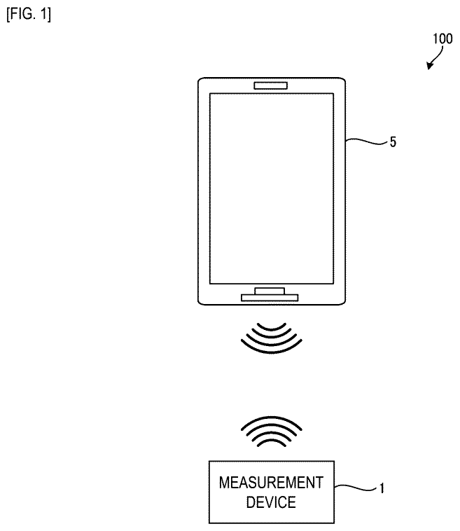

FIG. 1 illustrates an information management system 100 including a measurement device 1 of the present invention and an information terminal 5 that performs wireless communication with the measurement device 1.

The measurement device 1 includes a biological data measurement device that measures biological data such as a body weight, a body composition, blood pressure, a pulse, a heart rate, body temperature, blood glucose, or a blood oxygen saturation level. The measurement device 1 includes a measurement sensor for measuring a measurement target amount. The measurement target amount of the measurement sensor includes biological data such as a body weight, a body fat percentage, a blood pressure value, a pulse rate, a heart rate, body temperature, a blood glucose level, or a blood oxygen saturation level in accordance with the measurement device 1. The measurement device 1 is a non-wearable measurement device. The non-wearable measurement device is a measurement device that is not wearable. The wearable measurement device is a measurement device (e.g., an activity meter) carried by being worn on the body of a user. For example, the measurement device 1 (non-wearable measurement device) is a measurement device such as a scale, a body composition meter, a body weight-composition meter, or a sphygmomanometer that is used in a state of being installed on a ground or a table. The measurement device 1 transmits the measured biological data as measurement biological data of the user to the information terminal 5 by wireless communication.

The information terminal 5 stores the measurement biological data received from the measurement device 1 into a data storage unit in the information terminal 5. The information terminal 5 can perform wireless communication also with an external device other than the measurement device 1, and stores information acquired from the external device into the data storage unit in the information terminal 5. The information terminal 5 is an information processing device that analyzes various types of information acquired from the measurement device 1 and other external devices. The information terminal 5 is a terminal having a display such as, for example, a smartphone, a tablet terminal, a laptop computer, a desktop computer, or a wearable terminal. The information terminal 5 may be set so as to acquire measurement biological data from a specific measurement device 1. The specific measurement device 1 from which the measurement biological data is acquired may be registered in advance in the data storage unit of the information terminal 5.

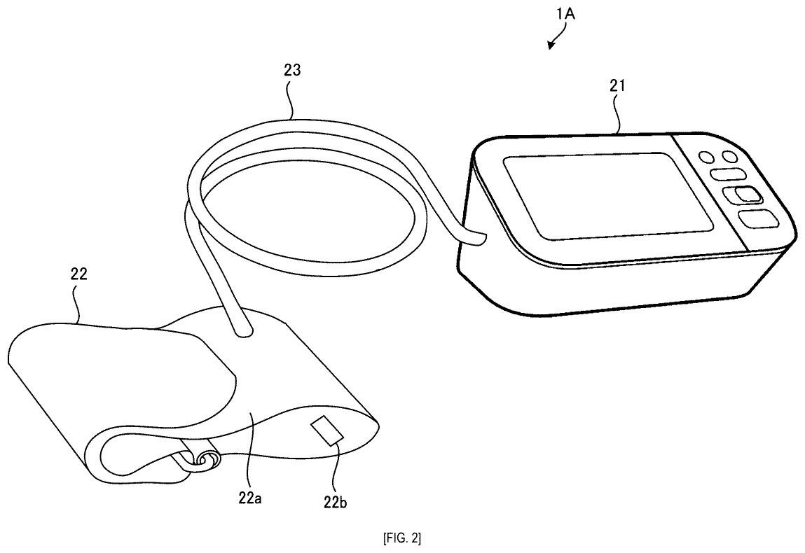

FIG. 2 is a view illustrating a sphygmomanometer 1A that is an example of the measurement device 1. The sphygmomanometer 1A is an example of a biological data measurement device, measures a blood pressure (pressure pulse wave data) of the user, and outputs a measurement result thereof to the user. The sphygmomanometer 1A transmits the measurement result as measurement biological data of the user to the information terminal 5 by wireless communication. For example, the sphygmomanometer 1A includes a main body portion 21, a cuff 22 that can be wound around a measurement target site (e.g., upper arm) of the user, and an air tube 23 that connects the main body portion 21 and the cuff 22. The cuff 22 includes a band-shaped body 22a attached around the upper arm and configured to change a compression pressure on the upper arm, and a sensor 22b disposed between the band-shaped body 22a and the upper arm. The measurement by the sphygmomanometer 1A includes a pressurization process of increasing the compression pressure and a depressurization process of decreasing the compression pressure after the pressurization process. Note that in the example of FIG. 2, the cuff 22 and the main body portion 21 are separated, but the cuff 22 may be integrated with the main body portion 21.

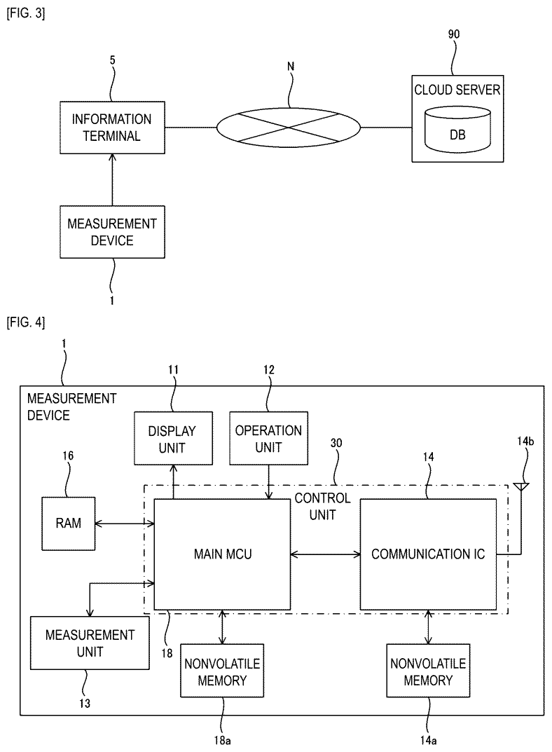

FIG. 3 is a view illustrating an example in which the information terminal 5 is connected to the network. As illustrated in FIG. 3, the information terminal 5 may be connected to a cloud server 90 via a wide area network N such as the Internet. The information terminal 5 may transmit the measurement biological data stored in the information terminal 5 to the cloud server 90 via the wide area network N, and the cloud server 90 may manage the measurement biological data of the user as a database. The information terminal 5 may acquire, via the wide area network N, the measurement biological data managed by the cloud server 90 and use the acquired measurement biological data.

### § 2 CONFIGURATION EXAMPLE

### Configuration of Measurement Device 1

FIG. 4 is a block diagram illustrating the configuration of the measurement device 1. The measurement device 1 includes a display unit 11 that can display various types of information, an operation unit 12 that can be operated by the user, a measurement unit 13 that measures biological data and the like, a communication integrated circuit (IC) 14 that performs communication with an external device, a nonvolatile memory 14a connected to the communication IC 14, and a communication antenna 14b. The measurement device 1 includes a random access memory (RAM) 16 that temporarily stores information, a main micro controller unit (MCU) 18 that controls the operation of the entire device, and a nonvolatile memory 18a connected to the main MCU 18. The main MCU 18 is an example of the first processor of the present invention. The communication IC 14 is an example of the second processor of the present invention. A combination of the main MCU 18 and the communication IC 14 is an example of a control unit 30 of the present invention. As a communication interface between the main MCU 18 and the communication IC 14, for example, an interface such as a UART is used.

The display unit 11 includes a liquid crystal display or an organic electro luminescence (EL) display, for example. The operation unit 12 is a user interface that receives a user operation such as a button or a touch panel. The button includes a button physically provided at the measurement device 1 or a virtual button displayed on the display unit 11.

The measurement unit 13 includes a sensor that measures biological data such as a body weight, a body composition, blood pressure, a pulse, a heart rate, body temperature, blood glucose, and a blood oxygen saturation level. What to be measured varies depending on a measurement target of the measurement device 1.

The nonvolatile memory 14a is a recording medium that stores a parameter necessary for implementing a predetermined function, a control recording medium, and biological data measured by the measurement unit 13. The nonvolatile memory 14a includes, for example, a flash memory. The nonvolatile memory 14a is provided with a biological data area allocated for storing biological data. The biological data stored in the nonvolatile memory 14a is managed by the communication IC 14.

The communication IC 14 implements a predetermined function by executing a control recording medium. For example, the communication IC 14 can perform near-field wireless communication by executing a communication recording medium stored in the nonvolatile memory 14a. The communication IC 14 performs communication in compliance with, for example, Bluetooth (registered trademark) Low Energy (BLE) standard. The communication IC 14 transmits, in a cyclic period, an advertise signal for performing wireless communication to an unspecified number of external devices by broadcast communication. The communication IC 14 sends an advertise signal including, for example, the name and attribute information of the measurement device 1. The BLE communication performed by the communication IC 14 is, for example, communication using a 2.4 GHz frequency.

By executing, for example, an erasure recording medium stored in the nonvolatile memory 18a, the communication IC 14 can erase an erasure target (e.g., biological data) written in the nonvolatile memory 14a. The communication IC 14 erases the biological data from the nonvolatile memory 14a in accordance with an erasure instruction signal transmitted from the main MCU 18 to the communication IC.

The communication IC 14 can manage the biological data by executing, for example, a management recording medium stored in the nonvolatile memory 14a. The biological data is biological data of the user measured by the measurement unit 13.

For example, the communication IC 14 performs write processing of writing the measured biological data into the nonvolatile memory 14a. The communication IC 14 performs read processing of reading the biological data from the nonvolatile memory 14a. The communication IC 14 writes the biological data into the nonvolatile memory 14a in accordance with a write instruction signal transmitted from the main MCU 18 to the communication IC, and reads the biological data from the nonvolatile memory 14a in accordance with a read instruction signal. The communication IC 14 performs write processing and read processing of the biological data with respect to the biological data area of the nonvolatile memory 14a. In the biological data area, information other than the biological data to be written in accordance with the write instruction signal from the main MCU 18 is not written. The biological data area is a dedicated area that can be used by the main MCU 18 in the area provided in the nonvolatile memory 14a.

The communication IC 14 performs transmission processing of transmitting the biological data read from the nonvolatile memory 14a to, for example, the information terminal 5 by wireless communication using the antenna 14b. The communication IC 14 performs transmission processing of the biological data in accordance with the transmission instruction signal transmitted from the main MCU 18 to the communication IC 14.

The RAM 16 includes a semiconductor device such as, for example, a dynamic RAM (DRAM) or a static RAM (SRAM), temporarily stores information, and also operates as a work area of the main MCU 18.

The nonvolatile memory 18a is a recording medium that stores a parameter necessary for implementing a predetermined function, a control recording medium, address information of a biological data area in the nonvolatile memory 14a connected to the communication IC 14, and the like. The nonvolatile memory 18a includes, for example, an electrically erasable programmable read only memory (EEPROM). Note that in the present example, the nonvolatile memory 14a has a configuration independent of the communication IC 14, but for example, the nonvolatile memory 14a may be one module with the communication IC 14.

The main MCU 18 implements a predetermined function by executing a control recording medium. For example, the main MCU 18 can perform measurement based on the biological data acquired by the measurement unit 13 by executing a measurement recording medium stored in the nonvolatile memory 18a.

By executing, for example, an erasure instruction recording medium stored in the nonvolatile memory 18a, the main MCU 18 can instruct erasure of the biological data written in the nonvolatile memory 14a. For example, the main MCU 18 specifies an address of a biological data area in the nonvolatile memory 14a, and transmits, to the communication IC 14, an erasure instruction signal for erasing the erasure target (e.g., biological data) from the nonvolatile memory 14a. For example, upon receiving an instruction for data measurement by pressing of a measurement button, the main MCU 18 starts measurement of the biological data and starts an instruction for data erasure of the nonvolatile memory 14a to the communication IC 14.

Until the data erasure in the communication IC 14 is completed, the main MCU 18 buffers the biological data obtained in the measurement. When the data erasure in the communication IC 14 is completed, the main MCU 18 transmits the buffered biological data to the communication IC 14 and writes the buffered biological data into the nonvolatile memory 14a. The measurement is to calculate a measurement value (e.g., blood pressure value) based on pressure pulse wave data obtained while increasing and pressurizing the compression pressure on the measurement target site and then depressurizing the compression pressure when the biological data of the measurement target is, for example, pressure pulse wave data. The data erasure is, for example, erasure of a certain data unit (one sector (e.g., 4 KB)). The buffering is to temporarily store the biological data obtained in the measurement into a different memory from the nonvolatile memory 14a. The different memory may be, for example, a volatile memory such as the RAM 16 in the main MCU 18 or a nonvolatile memory such as the nonvolatile memory 18a.

The main MCU 18 can manage and instruct the measured biological data by executing, for example, a management instruction recording medium stored in the nonvolatile memory 18a. For example, the main MCU 18 transmits, to the communication IC 14, a write instruction signal for specifying an address of a write destination in the biological data area of the nonvolatile memory 14a and writing the biological data into the nonvolatile memory 14a. The main MCU 18 transmits the biological data measured by the measurement unit 13 and buffered in the RAM 16 to the communication IC 14 together with the write instruction signal. The main MCU 18 sequentially transmits, to the communication IC 14, the biological data in which the biological data that is time-series data is divided at regular time intervals. For example, the main MCU 18 transmits the biological data to the communication IC in 18 bytes every 32 msec.

The main MCU 18 transmits, to the communication IC 14, a read instruction signal for specifying an address of a read source in the biological data area of the nonvolatile memory 14a and reading the biological data from the nonvolatile memory 14a. Note that the address specification of write and read may be, for example, specification of a start address of write and read and specification of a size of write information or read information in the biological data area of the nonvolatile memory 14a, or specification of a start address and an end address of write and read.

The main MCU 18 can instruct the transmission processing of the communication IC 14 by executing, for example, a transmission instruction recording medium stored in the nonvolatile memory 18a. For example, the main MCU 18 transmits, to the communication IC 14, a transmission instruction signal for transmitting an advertise signal for wireless communication (BLE communication) in a cyclic period, and a transmission instruction signal for transmitting biological data read from the nonvolatile memory 14a to an external device such as the information terminal 5.

When writing the measured biological data into the nonvolatile memory 14a, when reading the biological data, and when transmitting the biological data to the external device, the main MCU 18 only performs processing of transmitting, to the communication IC 14, an instruction signal including address specification of the nonvolatile memory 14a. The write processing of the biological data to the nonvolatile memory 14a, the read processing of the biological data, and the transmission processing of the biological data to the external device are executed by the communication IC 14 having received an instruction from the main MCU 18. That is, the main MCU 18 is configured to be indirectly accessible to the nonvolatile memory 14a via the communication IC 14, but not directly accessible to the nonvolatile memory.

Before sensing of the biological data, the main MCU 18 transmits, to the communication IC 14, a saving start instruction signal that instructs starting of saving of the biological data into the nonvolatile memory 14a. After sensing of the biological data, the main MCU 18 transmits, to the communication IC 14, a saving end instruction signal that instructs ending of the saving of the biological data into the nonvolatile memory 14a. After the sensing of the biological data ends, the main MCU 18 receives, from the communication IC 14, result information regarding writing of the biological data into the nonvolatile memory 14a. The result information is transmitted by being included in a response signal from the communication IC 14 to the main MCU 18 in response to the instruction of ending the saving. The result information includes information indicating the number of receptions of the biological data that the communication IC 14 has received from the main MCU 18 and information indicating the number of failures of the biological data that the communication IC 14 has failed to write into the nonvolatile memory 14a. Note that the result information may be information indicating the number of successes of the biological data successfully written into the nonvolatile memory 14a.

By executing, for example, an information output recording medium stored in the nonvolatile memory 18a, the main MCU 18 outputs a biological measurement result based on measured biological data, for example, a blood pressure measurement result based on pressure pulse wave data. The main MCU 18 displays the blood pressure measurement result on a screen of the display unit 11 of the measurement device 1, for example. The main MCU 18 may output the blood pressure measurement result by voice from the measurement device 1 or may wirelessly transmit the blood pressure measurement result to the information terminal 5.

### Configuration of Information Terminal 5

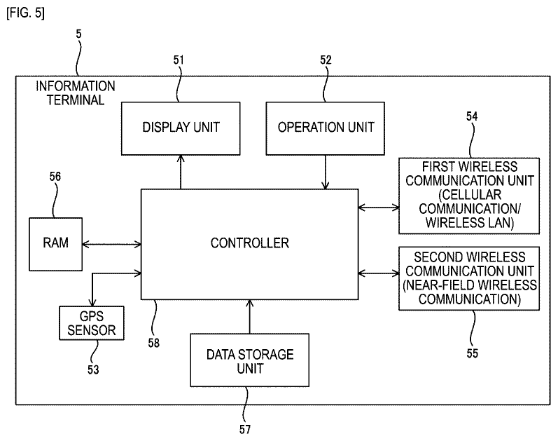

FIG. 5 is a block diagram illustrating the configuration of the information terminal 5. The information terminal 5 includes a display unit 51 that can display various types of information, an operation unit 52 than can be operated by the user, a global positioning system (GPS) sensor 53 for detecting a position, and a first wireless communication unit 54 and a second wireless communication unit 55 that perform communication with an external device. The information terminal 5 includes a RAM 56 that temporarily stores information, a data storage unit 57 that stores information and programs, and a controller 58 that controls the operation of the entire terminal.

The display unit 51 includes a liquid crystal display or an organic electro luminescence (EL) display, for example. The operation unit 52 is a user interface that receives a user operation such as a button or a touch panel. The button includes a button physically provided at the information terminal 5 or a virtual button displayed on the display unit 51. The GPS sensor 53 is a sensor for detecting the current position of the information terminal 5.

The first wireless communication unit 54 is a communication unit that performs cellular communication, for example, a circuit (module) that can perform communication in compliance with a standard such as 4G, 5G, or long term evolution (LTE: registered trademark). The first wireless communication unit 54 is a communication unit that performs wireless LAN communication, for example, a circuit that can perform communication in compliance with a standard such as Wi-Fi (registered trademark). The second wireless communication unit 55 is a communication unit that performs near-field wireless communication, for example, a circuit (module) for performing communication in compliance with the BLE standard.

The second wireless communication unit 55 acquires the biological data of the user measured by the measurement device 1, for example, by performing BLE communication with the communication IC 14 of the measurement device 1. The second wireless communication unit 55 receives the advertise signal transmitted from the communication IC 14 of the measurement device 1 by scanning. The second wireless communication unit 55 recognizes the measurement device 1 from the received advertise signal, and transmits a connection request to the measurement device 1 when communication connection is desired. Note that the measurement device 1 waits for a connection request for a predetermined time after transmitting the advertise signal, stops sending the advertise signal upon receiving the connection request within the predetermined time, and switches to one-to-one connection communication with a connection request partner.

The RAM 56 includes a semiconductor device such as a DRAM or an SRAM, for example, temporarily stores information, and operates as a work area of the controller 58.

The data storage unit 57 is a recording medium that stores a parameter necessary for implementing a predetermined function, a control recording medium, measurement biological data acquired from the measurement device 1, and the like. The data storage unit 57 includes, for example, a hard disk drive (HDD) or a semiconductor storage device (SSD).

The controller 58 implements a predetermined function by executing the control recording medium. Note that in the present embodiment, for example, management application software for an information terminal is installed in advance as a control recording medium in the data storage unit 57, and the controller 58 implements the predetermined function by executing this management application software. For example, when the management application software for the information terminal is started, the controller 58 controls the second wireless communication unit 55 so as to receive the advertise signal by scanning. When the advertise signal from the measurement device 1 is received, the controller 58 transmits a connection request to the measurement device 1 and controls the second wireless communication unit 55 so as to acquire the measurement biological data from the measurement device 1.

### § 3 OPERATION EXAMPLE

### Operation Example of Measurement Device 1

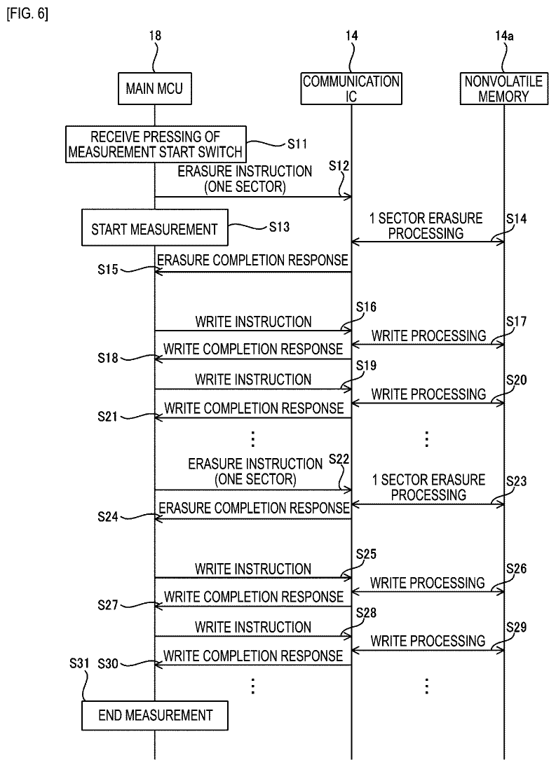

Next, an operation example of the measurement device 1 will be described with reference to FIG. 6. FIG. 6 is a sequence diagram illustrating operations of the main MCU 18, the communication IC 14, and the nonvolatile memory 14a in the measurement device 1. Note that the present example will be described below with the measurement device 1 being the sphygmomanometer 1A, and the biological data measured by the sphygmomanometer 1A being pressure pulse wave data.

It is assumed that an upper arm of the user is attached with the cuff 22 of the sphygmomanometer 1A and a switch for starting measurement is pressed.

First, the main MCU 18 receives pressing of the measurement start switch (step S11). Next, in response to the pressing of the measurement start switch, the main MCU 18 transmits, to the communication IC 14, an erasure instruction signal for erasing data in the biological data area in the nonvolatile memory 14a (step S12).

The erasure instruction signal includes an address of the nonvolatile memory 14a in which data is erased and a data unit thereof (e.g., one sector (4 KB)). In response to the pressing of the measurement start switch, the main MCU 18 starts measurement of the pressure pulse wave data by the sphygmomanometer 1A (step S13). The main MCU 18 first pressurizes the cuff 22 to sense the pressure pulse wave data, and buffers the sensed pressure pulse wave data into the RAM 16. Note that the measurement by the sphygmomanometer 1A includes a pressurization process of increasing the compression pressure and a depressurization process of decreasing the compression pressure after the pressurization process. The main MCU 18 measures the pressure pulse wave data in order of the pressurization process and then the depressurization process.

Next, in response to the erasure instruction signal received in step S12, the communication IC 14 performs processing of erasing data of one sector in the instructed area (step S14). The erasure processing for each sector is that, for example, when viewed from the communication IC 14 side, the communication IC 14 first transmits an erasure instruction for each sector to the nonvolatile memory 14a, and receives, from the nonvolatile memory 14a, a response indicative of having responded the erasure instruction (response indicating that one sector has been erased). Next, the communication IC 14 transmits, to the nonvolatile memory 14a, a read request for reading the erased one sector, receives, from the nonvolatile memory 14a, a response (read data of one sector) corresponding to the read request, and then ends by verification. Next, the communication IC 14 transmits, to the main MCU 18, a response signal notifying that the erasure processing is completed (step S15). The response signal of erasure completion includes, for example, a result code indicating that the erasure processing is completed, an address and a data unit of the erased data in the nonvolatile memory 14a.

Next, upon receiving the response signal of erase completion in step S15, the main MCU 18 transmits, to the communication IC 14, a write instruction signal for writing the measured pressure pulse wave data into the biological data area of the nonvolatile memory 14a (step S16). The write instruction signal includes an address and a data size of the nonvolatile memory 14a into which the pressure pulse wave data is written, and the pressure pulse wave data to be written. The pressure pulse wave data is transmitted by dividing, for example, at regular time intervals, the pressure pulse wave data measured by the measurement unit 13. The pressure pulse wave data to be written is transmitted from the pressure pulse wave data sensed in the pressurization process. That is, the pressure pulse wave data is sequentially transmitted from the data buffered into the RAM 16 from step S13.

However, pressure pulse wave data measured before the transmission to the communication IC 14 is started, the pressure pulse wave data overflowing from the buffer area of the RAM 16 without being completely buffered, for example, is not written into the nonvolatile memory 14a and is discarded.

Next, in response to the write instruction signal received in step S16, the communication IC 14 performs write processing of writing the pressure pulse wave data transmitted from the main MCU 18 to a specified address in the biological data area of the nonvolatile memory 14a (step S17). The write processing is that, for example, when viewed from the communication IC 14 side, the communication IC 14 first transmits, to the nonvolatile memory 14a, a write instruction for pressure pulse wave data transmitted from the main MCU 18, and receives, from the nonvolatile memory 14a, a response indicative of having responded the write instruction (response indicating that the pressure pulse wave data has been written). Next, the communication IC 14 transmits, to the nonvolatile memory 14a, a read request for reading the written pressure pulse wave data, receives, from the nonvolatile memory 14a, a response (read pressure pulse wave data) corresponding to the read request, and then ends by verification. Next, the communication IC 14 transmits, to the main MCU 18, a response signal notifying that the write processing of the pressure pulse wave data is completed (step S18). The response signal of write completion includes, for example, a result code indicating that the write processing is completed, an address and a data size of the written data in the nonvolatile memory 14a.

Next, upon receiving the response signal of write completion in step S18, the main MCU 18 transmits, to the communication IC 14, a write instruction signal for writing the measured next pressure pulse wave data into the biological data area of the nonvolatile memory 14a (step S19). As described above, the next pressure pulse wave data is the next pressure pulse wave data in the pressure pulse wave data measured in order of the pressurization process and then the depressurization process. The other processing in step S19 is similar to the processing of the write instruction in step S16.

Next, in response to the write instruction signal received in step S19, the communication IC 14 performs write processing of writing the next pressure pulse wave data transmitted from the main MCU 18 to a specified address in the biological data area of the nonvolatile memory 14a (step S20). The processing in step S20 is similar to the write processing in step S17 except that the pressure pulse wave data to be written is the next pressure pulse wave data. Next, the communication IC 14 transmits, to the main MCU 18, a response signal notifying that the write processing of the next pressure pulse wave data is completed (step S21).

Similarly, processing of a write instruction, processing of write, and response processing of write completion are repeatedly executed among the main MCU 18, the communication IC 14, and the nonvolatile memory 14a. While the processing of a write instruction, the processing of write, and the response processing of write completion are repeated, the main MCU 18 continues the measurement of the pressure pulse wave data, and performs the depressurization process after the pressurization process is ended.

Next, when the main MCU 18 compares the write data amount of the pressure pulse wave data for which the write instruction has been issued with the area (one sector (4 KB)) of the nonvolatile memory 14a erased in step S14 and determines that the free area of the nonvolatile memory 14a is insufficient (data amount close to 4 KB has been written), an erasure instruction signal for erasing the next one sector in the biological data area of the nonvolatile memory 14a is transmitted to the communication IC 14 (step S22).

Next, in response to the erasure instruction signal received in step S22, the communication IC 14 performs processing of erasing data of the next one sector in the instructed area (step S23). The processing in step S23 is similar to the erasure processing in step S14 except that the area to be erased is the next one sector. Next, the communication IC 14 transmits, to the main MCU 18, a response signal notifying that the erasure processing is completed (step S24). At this time, when the measurement of the pressure pulse wave data shifts from the measurement of the pressurization process to the measurement of the depressurization process in the middle of memory erasure processing from step S22 to step S24, for example, the measurement of the depressurization process needs not be started and may be brought into a standby state until the memory erasure processing is ended.

Next, similarly to the case described in steps S19 to S21 above, for example, the processing of a write instruction, the processing of write, and the response processing of write completion are repeated until the measurement of the present pressure pulse wave data is ended as in steps S25 to S27 and steps S28 to S30, and the like.

When the measurement of the pressure pulse wave data in the depressurization process is completed, the main MCU 18 ends the present measurement (step S31).

Note that in the measurement of the pressure pulse wave data described above, memory erasure is performed for each sector in the memory erasure processing (e.g., steps S12, S14, and S15, steps S22 to S24, and the like), but no such limitation is intended. For example, the memory erasure processing may be performed by performing memory erasure of a necessary size in first steps S12, S14, and S15.

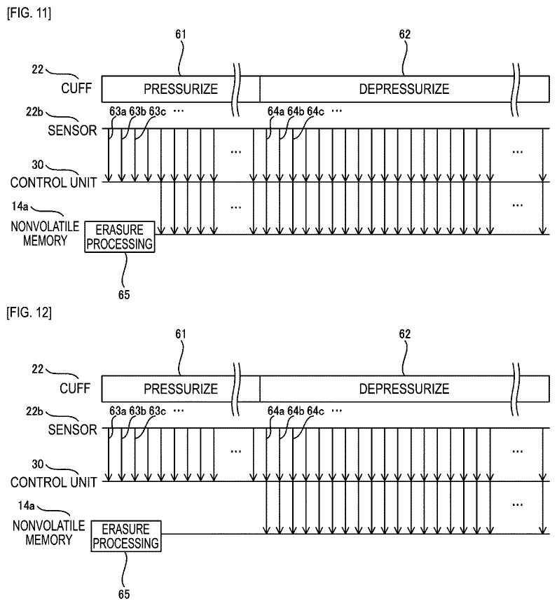

In the measurement of the pressure pulse wave data described above, the case where the sensing data of the pressurization process and the depressurization process are buffered and saved in the nonvolatile memory 14a has been described, but no such limitation is intended. For example, the sensing data in the pressurization process may be discarded without being buffered and saved into the nonvolatile memory 14a. This point will be described later with reference to FIGS. 11 and 12.

In the measurement of the pressure pulse wave data described above, the case where the sensing data is buffered and saved in the nonvolatile memory 14a has been described, but no such limitation is intended. For example, in a case where data can be written into the nonvolatile memory 14a, the sensing data may be sequentially written to the nonvolatile memory 14a without being buffered, or the buffered sensing data may be immediately read from the buffer and written into the nonvolatile memory 14a while the sensing data is constantly buffered.

In the measurement of the pressure pulse wave data described above, before the erasure instruction in step S12, saving start processing for starting saving of the sensing data into the nonvolatile memory 14a may be performed. After the measurement processing in step S31, saving end processing for ending saving of the sensing data into the nonvolatile memory 14a may be performed.

The transmission processing for transmitting the pressure pulse wave data written into the nonvolatile memory 14a from the sphygmomanometer 1A to the external information terminal 5 is performed, for example, after the measurement of the pressure pulse wave data described above ends. In this case, the main MCU 18 transmits, to the communication IC 14, a transmission instruction signal for transmitting the pressure pulse wave data. The transmission instruction signal includes specification of an address in the nonvolatile memory 14a of the pressure pulse wave data to be transmitted and the size thereof. Next, the communication IC 14 reads the pressure pulse wave data from the nonvolatile memory 14a in accordance with the transmission instruction signal received from the main MCU 18, and transmits the read pressure pulse wave data to the information terminal 5 by wireless communication. The communication IC 14 transmits, to the information terminal 5, result information related to writing of the pressure pulse wave data together with the pressure pulse wave data. The result information may include, for example, the number of received pressure pulse wave data received by the communication IC 14 from the main MCU 18 and the number of failed pressure pulse wave data that has failed to be written to the nonvolatile memory 14a.

### § 4 MEASUREMENT DATA EXAMPLE

### Measurement Data of Measurement Device 1

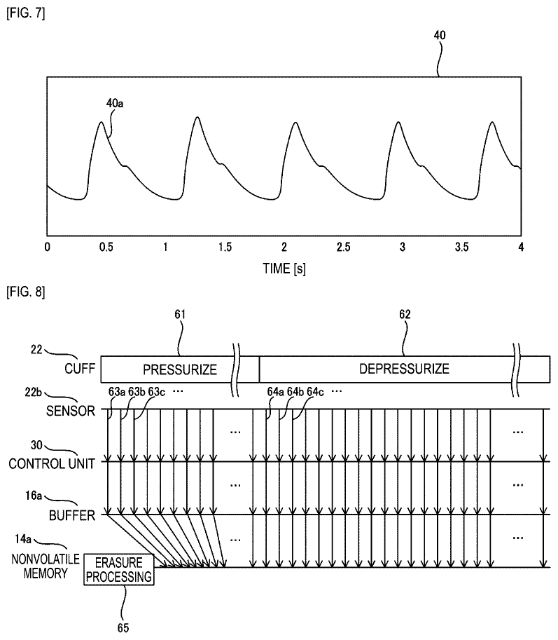

FIG. 7 is a view illustrating an example of biological data to be measured by the measurement device 1. The present example illustrates an example of pressure pulse wave data to be measured by the sphygmomanometer 1A. As illustrated in FIG. 7, pressure pulse wave data 40 is measured as a continuous conduction wave 40a having substantially constant cyclicity. The pressure pulse wave data 40 having been measured is transmitted from the main MCU 18 to the communication IC. The main MCU 18 divides, at regular time intervals, and sequentially transmits, to the communication IC 14, the pressure pulse wave data 40 having been measured.

### § 5 FLOW OF MEASUREMENT DATA

### Example of First Flow of Measurement Data

FIG. 8 is a view illustrating an example of the first flow of pressure pulse wave data measured by the sphygmomanometer 1A. As illustrated in FIG. 8, in the example of the first flow, in a pressurization process 61 and a depressurization process 62 of measurement, the sensing data 63a, 63b, 63c, . . . , and 64a, 64b, 64c, . . . are acquired by the sensor 22b of the cuff 22. When erasure processing 65 of erasing the biological data area of the nonvolatile memory 14a is started by the control unit 30, measurement of the pressurization process 61 is started.

Since this erasure processing 65 is started, the sensing data 63a, 63b, 63c, . . . acquired in the pressurization process 61 in the period of the erasure processing 65 are sequentially buffered into the buffer 16a of the RAM 16 by the control unit 30 until this erasure processing 65 is completed. Then, when the erasure processing 65 of the nonvolatile memory 14a is completed, the buffered sensing data 63a, 63b, 63c, . . . are sequentially written into the nonvolatile memory 14a by the control unit 30.

In the example of the first flow, in parallel with the erasure processing 65, all the sensing data 63a, 63b, 63c, . . . acquired in the pressurization process 61 are buffered without overflowing from the buffer 16a, and the buffered data are sequentially written into the nonvolatile memory 14a without being discarded. In the example of the first flow, all the sensing data 64a, 64b, 64c, . . . acquired in the depressurization process 62 are also sequentially written into the nonvolatile memory 14a without being discarded. The sensing data 64a, 64b, 64c, . . . are immediately read and written into the nonvolatile memory 14a when buffered in the buffer 16a.

Note that the present example assumes a configuration in which the erasure processing 65 of the nonvolatile memory 14a is performed only once together with the start of the pressurization process 61. However, for example, in a case where the free area of the nonvolatile memory 14a having been erased becomes insufficient, the erasure processing 65 may be performed even in the middle of the depressurization process 62.

### Example of Second Flow of Measurement Data

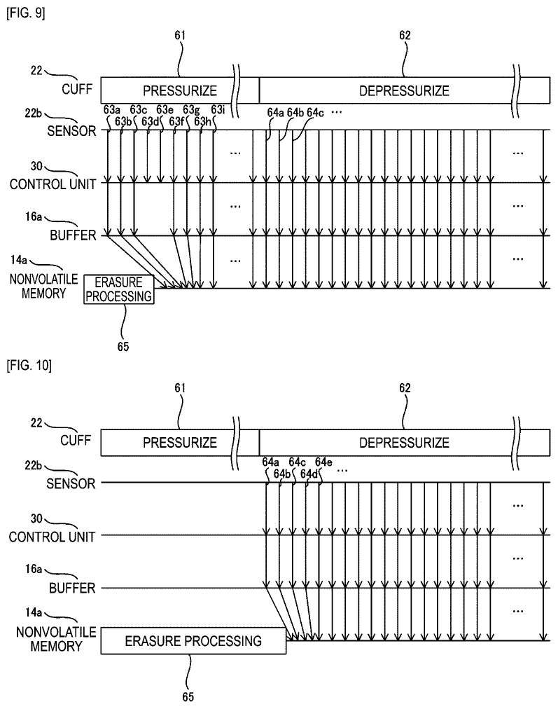

FIG. 9 is a view illustrating an example of the second flow of pressure pulse wave data measured by the sphygmomanometer 1A. The example of the second flow is an example of a case where the acquired sensing data cannot fully put into the buffer 16a of the RAM 16 and part of the sensing data is discarded. As illustrated in FIG. 9, in the example of the second flow, similarly to the example of the first flow, in the pressurization process 61 and the depressurization process 62 of measurement, the sensing data 63a, 63b, 63c, . . . , and 64a, 64b, 64c, . . . are acquired by the sensor 22b of the cuff 22. Similarly to the example of the first flow, when the erasure processing 65 of the nonvolatile memory 14a is started, the measurement of the pressurization process 61 is started, and the sensing data 63a, 63b, 63c, . . . acquired in the pressurization process 61 are sequentially buffered into the buffer 16a.

However, in the example of the second flow, the sensing data 63a, 63b, 63c, . . . in the pressurization process 61 acquired in parallel with the erasure processing 65 of the nonvolatile memory 14a cannot fully put into the buffer 16a of the RAM 16, and part of the sensing data 63d and 63e overflow from the buffer 16a and are not buffered. Therefore, the sensing data 63d and 63e acquired in the pressurization process 61 are not written to the nonvolatile memory 14a but are discarded. Note that the sensing data other than the sensing data 63d and 63e acquired in the pressurization process 61 are buffered in the buffer 16a without overflowing and sequentially written into the nonvolatile memory 14a. The sensing data 64a, 64b, 64c, . . . acquired in the depressurization process 62 are buffered in the buffer 16a without overflowing, and sequentially written into the nonvolatile memory 14a, similarly to the example of the first flow.

### Example of Third Flow of Measurement Data

FIG. 10 is a view illustrating an example of the third flow of pressure pulse wave data measured by the sphygmomanometer 1A. The example of the third flow is an example of a case where sensing data is not acquired in the pressurization process of measurement. As illustrated in FIG. 10, in the example of the third flow, the sensing data 64a, 64b, 64c, . . . are acquired by the sensor 22b in the depressurization process 62 of measurement. When the pressurization process 61 is started, the erasure processing 65 of erasing the biological data area of the nonvolatile memory 14a is started.

Note that the example of the third flow assumes that the erasure processing 65 takes more time than that in the examples of the first and second flows, and the period of the erasure processing 65 is performed in parallel with a part of the first period of the depressurization process 62. Therefore, the sensing data acquired in parallel with the period of the erasure processing 65 of the sensing data acquired in the depressurization process 62 is buffered into the buffer 16a until this erasure processing 65 is completed. By this, in the example of the third flow, as illustrated in FIG. 10, part of sensing data 64a, 64b, 64c, and 64d are not immediately read from the buffer 16a but sequentially written into the nonvolatile memory 14a with a delay of a buffered period. Note that data of the sensing data 64e and thereafter are immediately read and written into the nonvolatile memory 14a when buffered in the buffer 16a.

As described above, upon receiving the measurement instruction, the measurement device 1 starts measurement of the biological data and data erasure of the nonvolatile memory 14a, buffers into, for example, the RAM 16, the biological data obtained in the measurement until the data erasure of the nonvolatile memory 14a is completed, and writes, into the nonvolatile memory 14a, the biological data buffered into the RAM 16 when the data erasure of the nonvolatile memory 14a is completed. According to this configuration, the measurement based on the biological data is executed in parallel with the data erasure of the nonvolatile memory 14a, whereby the measurement can be started without waiting for completion of the data erasure of the nonvolatile memory 14a. This can shorten the time from the reception of the measurement instruction to the start of the measurement, and can shorten the waiting time of the user. This can suppress deterioration of usability that it is necessary to secure an erased area for saving large-capacity biological data before measurement of the biological data while saving the large-capacity biological data into the nonvolatile memory 14a.

The measurement device 1 is the sphygmomanometer 1A, and includes the pressurization process of increasing the compression pressure of the cuff 22 and the depressurization process of decreasing the compression pressure of the cuff 22 after the pressurization process. In the measurement of the pressure pulse wave data by the sphygmomanometer 1A, the pressurization process is first executed, and the sensing data between the pressurization processes is not so important as compared with the sensing data between the depressurization processes to be executed thereafter. Therefore, even if the measurement is started while the data erasure of the nonvolatile memory 14a is performed and the sensing data at the beginning of the measurement is lost because the sensing data cannot be saved in the buffer, the sensing data in the pressurization process is missed, and therefore the influence on the data analysis and the like is small. Therefore, the measurement device 1 is suitable for measurement of pressure pulse wave data by the sphygmomanometer 1A. However, depending on the measurement algorithm of pressure pulse wave data, the sensing data between the pressurization processes is also important, and therefore in that case, it is preferable to buffer the sensing data between the pressurization processes as much as possible and save it in the nonvolatile memory 14a.

In the measurement device 1, the main MCU 18 transmits the biological data to the communication IC 14 to write the biological data into the nonvolatile memory 14a, and the communication IC 14 transmits, to the information terminal 5, the biological data written into the nonvolatile memory 14a. In the configuration in which measurement based on the biological data is executed in parallel with data erasure of the nonvolatile memory 14a, if these processings are performed by one processor, a processing load increases, and a delay occurs in the processing such as the measurement. Therefore, by providing the communication IC 14 that performs erasure of the nonvolatile memory 14a, writing to the nonvolatile memory, and wireless communication separately from the main MCU 18 that performs measurement, the processing load for erasing the data of the nonvolatile memory 14a, writing the pressure pulse wave data to the nonvolatile memory 14a, and transmitting the pulse wave data to the information terminal 5 can be distributed to the communication IC 14, and the processing load of the main MCU 18 can be reduced. This can suppress a delay in processing such as measurement by the main MCU 18.

The main MCU 18 of the measurement device 1 specifies an address of a write destination in the biological data area of the nonvolatile memory 14a and causes the communication IC 14 to write the biological data into the nonvolatile memory 14a, and specifies an address of a read source in the biological data area of the nonvolatile memory 14a and causes the communication IC 14 to read the biological data from the nonvolatile memory 14a and transmit the biological data read from the nonvolatile memory 14a to an external device such as the information terminal 5 by wireless communication. According to this configuration, since the main MCU 18 is configured to specify the address of the nonvolatile memory 14a connected to the communication IC 14, and instruct the communication IC 14 to erase data and write, read, and transmit biological data, flow control is unnecessary in the interface between the main MCU 18 and the communication IC 14, and the transfer speed of the biological data to the information terminal 5 can be improved. Since the communication IC 14 may perform data erasure of a specified address in the nonvolatile memory 14a, writing of information to the specified address in the nonvolatile memory 14a, and reading and transmission of information from the specified address in the nonvolatile memory 14a, the communication IC 14 can have a simple configuration. By an instruction to the communication IC 14, the main MCU 18 can flexibly perform data erasure of the nonvolatile memory 14a, writing of biological data to the nonvolatile memory 14a, reading of biological data from the nonvolatile memory 14a, and transmission of the read biological data. However, since it is not necessary for the main MCU 18 itself to perform processing with a large load such as erasure processing, write processing, read processing, and transmission processing of biological data and the like, it is possible to reduce the processing load of the main MCU 18 as described above.

The measurement device 1 has a biological data area allocated for storing biological data in the nonvolatile memory 14a. The biological data stored in the biological data area is configured to be managed by the communication IC 14 and not accessible from the main MCU 18. According to this configuration, since information other than the biological data is not written into the biological data area, it is possible to suppress interference between writing of the biological data by an instruction from the main MCU 18 to the communication IC 14 and writing of other information by the communication IC 14. As compared with a configuration in which the main MCU 18 and the communication IC 14 share one nonvolatile memory, access processing can be distributed and sped up.

### § 5 MODIFIED EXAMPLES

While an embodiment of the present invention has been described in detail above, the above description is merely illustrative of the present invention in all respects. Various modifications and variations can be made without departing from the scope of the present invention. For example, the following changes are possible. Note that in the following, the same reference signs are used for components that are the same as those of the above-described embodiment, and descriptions thereof are omitted as appropriate. The following modified examples can be combined as appropriate.

In the above embodiment, when the biological data measured by the main MCU 18 is written into the nonvolatile memory 14a and when it is read from the nonvolatile memory 14a, the measured biological data is transmitted to the communication IC 14 by specifying the addresses of the write destination and the read source in the nonvolatile memory 14a, but no such limitation is intended. For example, the main MCU 18 may transmit the biological data to the communication IC 14 without specifying the addresses of the write destination and the read source of the biological data in the nonvolatile memory 14a.

In the above embodiment, when the main MCU 18 transmits the biological data to the communication IC 14 and causes the biological data to be written into the nonvolatile memory 14a, a response signal (delivery confirmation) of write completion is received from the communication IC 14 every time the biological data is transmitted, but no such limitation is intended. For example, the main MCU 18 may sequentially transmit and write the biological data to the communication IC 14 without performing delivery confirmation (e.g., in a streaming manner). This can improve the transfer speed of the biological data from the main MCU 18 to the communication IC 14. The processing load of the main MCU 18 during sensing can be reduced.

In the above embodiment, the configuration in which the biological data is pulse wave data and the pressure pulse wave data is acquired as pulse wave data has been described, but the measurement device 1 may be configured to measure volume pulse wave data as pulse wave data.

In the above embodiment, the nonvolatile memory 14a has been described as the memory connected to the communication IC 14 (second processor), but the memory connected to the communication IC 14 (second processor) is not limited to the nonvolatile memory 14a and may be a volatile memory or the like.

In the above embodiment, the configuration in which the pulse wave data is saved in the nonvolatile memory 14a connected to the communication IC 14 has been described, but the saving destination of the pulse wave data is not limited to the nonvolatile memory 14a, and may be, for example, the nonvolatile memory 18a connected to the main MCU 18.

In the measurement of the pressure pulse wave data described above, the case where the sensing data of the pressurization process and the depressurization process are buffered and saved in the nonvolatile memory 14a has been described, but no such limitation is intended. For example, as illustrated in FIGS. 11 and 12, the control unit 30 may save the sensing data into the nonvolatile memory 14a without performing the buffering.

FIG. 11 is a view illustrating an example of the fourth flow of pressure pulse wave data measured by the sphygmomanometer 1A. As illustrated in FIG. 11, in the example of the fourth flow, in the pressurization process 61 and the depressurization process 62 of measurement, the sensing data 63a, 63b, 63c, . . . , and 64a, 64b, 64c, . . . are acquired by the sensor 22b of the cuff 22. When erasure processing 65 of erasing the biological data area of the nonvolatile memory 14a is started by the control unit 30, measurement of the pressurization process 61 is started.

Since this erasure processing 65 is started, the sensing data 63a, 63b, 63c, . . . acquired in the pressurization process 61 in the period of the erasure processing 65 are discarded by the control unit 30. Then, the sensing data acquired in the pressurization process 61 after the period of the erasure processing 65 is sequentially written into the nonvolatile memory 14a by the control unit 30.

That is, in the example of the fourth flow, the sensing data 63a, 63b, 63c, . . . acquired in the pressurization process 61 in the period of the erasure processing 65 are discarded, and the sensing data acquired in the pressurization process 61 after the period of the erasure processing 65 and the sensing data 64a, 64b, 64c, and 64d acquired in the depressurization process 62 are sequentially written into the nonvolatile memory 14a without being discarded. In this manner, the control unit 30 may discard the sensing data 63a, 63b, 63c, . . . acquired in the period of the erasure processing 65, and save, into the nonvolatile memory 14a, the sensing data acquired after completion of the erasure processing 65. Note that in the example of FIG. 11, the sensing data 63a, 63b, 63c, . . . are acquired by the sensor 22b also during the erasure processing 65, but the sensor 22b needs not be operated in the period of the erasure processing 65.

FIG. 12 is a view illustrating an example of the fifth flow of pressure pulse wave data measured by the sphygmomanometer 1A. As illustrated in FIG. 12, in the example of the fifth flow, in the pressurization process 61 and the depressurization process 62 of measurement, the sensing data 63a, 63b, 63c, . . . , and 64a, 64b, 64c, . . . are acquired by the sensor 22b of the cuff 22. When erasure processing 65 of erasing the biological data area of the nonvolatile memory 14a is started by the control unit 30, measurement of the pressurization process 61 is started.

The sensing data 63a, 63b, 63c, . . . acquired in the pressurization process 61 are discarded by the control unit 30. The sensing data 64a, 64b, 64c, and 64d acquired in the depressurization process 62 are sequentially written into the nonvolatile memory 14a by the control unit 30. In this manner, the control unit 30 may discard the sensing data 63a, 63b, 63c, . . . acquired in the pressurization process 61, and save the sensing data 64a, 64b, 64c, and 64d acquired in the depressurization process 62 into the nonvolatile memory 14a. Note that in the example of FIG. 12, the sensing data 63a, 63b, 63c, . . . are acquired by the sensor 22b also during the pressurization process 61, but the sensor 22b needs not be operated in the period of the pressurization process 61.

While various embodiments have been described above, needless to say, the present invention is not limited to such examples. It will be apparent to those skilled in the art that various changes and modifications can be made within the scope of the claims, and it should be understood that such changes and modifications also fall within the technical scope of the present invention. Components in the above-described embodiment may be combined as desired without departing from the gist of the invention.

The present application is based on Japanese Patent Application (No. 2023-035939) filed on Mar. 8, 2023, the contents of which are incorporated herein by reference.

### REFERENCE SIGNS LIST

1 Measurement device
1A Sphygmomanometer
5 Information terminal
13 Measurement unit
14 Communication IC (second processor)
14a, 18a Nonvolatile memory
16 RAM
18 Main MCU (first processor)
22 Cuff
22a Band-shaped body
22b Sensor
30 Control unit
40 Pressure pulse wave data
100 Information management system

1. A measurement device comprising:
a control unit configured to perform measurement using a sensor configured to acquire biological data; and
a memory, wherein
the control unit
starts the measurement and data erasure of the memory upon receiving an instruction for the measurement, and
writes biological data obtained in the measurement to the memory upon completing the data erasure.

2. The measurement device according to claim 1, wherein
the memory is a flash memory.

3. The measurement device according to claim 1, wherein
the biological data is pulse wave data.

4. The measurement device according to claim 3, wherein
the control unit outputs a blood pressure measurement result based on the pulse wave data.

5. The measurement device according to claim 3 comprising:
a cuff including a band-shaped body attached around a measurement target site and configured to change a compression pressure on the measurement target site, and the sensor disposed between the band-shaped body and the measurement target site, wherein
the measurement includes a pressurization process of increasing the compression pressure and a depressurization process of decreasing the compression pressure after the pressurization process.

6. The measurement device according to claim 5, wherein
the control unit does not write, into the memory, biological data obtained in the pressurization process, and writes, into the memory, biological data obtained in the depressurization process.

7. The measurement device according to claim 5, wherein
the control unit does not write, into the memory, biological data obtained before completion of the data erasure in the pressurization process, and writes, into the memory, biological data obtained after completion of the data erasure in the pressurization process and biological data obtained in the depressurization process.

8. The measurement device according to claim 1, wherein
the control unit
buffers biological data obtained in the measurement until the data erasure is completed, and
writes, into the memory, the biological data buffered upon completing the data erasure.

9. The measurement device according to claim 1, wherein
the control unit transmits, to an information terminal, the biological data written in the memory.

10. The measurement device according to claim 9, wherein
the control unit includes
a first processor that performs the measurement, and
a second processor that performs wireless communication with the information terminal,
the memory is connected to the second processor,
the first processor transmits the biological data to the second processor and writes the biological data into the memory, and
the second processor transmits, to the information terminal, the biological data written in the memory.

11. The measurement device according to claim 10, wherein
the first processor
specifies an address of a write destination in the memory and causes the second processor to write the biological data into the memory, and
specifies an address of a read source in the memory and causes the second processor to read the biological data from the memory and transmit the biological data to the information terminal.

12. The measurement device according to claim 10, wherein
the first processor specifies an address of an erasure target in the memory and causes the second processor to erase the biological data from the memory.

13. The measurement device according to claim 11, wherein
the memory includes an area allocated for the biological data, and
the address of the write destination and the address of the read source are addressed in the area.

14. The measurement device according to claim 10, wherein
the memory is inaccessible from the first processor.

15. A control method for a measurement device including a control unit that performs measurement using a sensor that acquires biological data, and a memory, wherein
the control unit
starts the measurement and data erasure of the memory upon receiving an instruction for the measurement, and
writes biological data obtained in the measurement to the memory upon completing the data erasure.

16. A control recording medium of a measurement device including a control unit that performs measurement using a sensor that acquires biological data, and a memory, the control recording medium for causing
the control unit to execute processing of
starting the measurement and data erasure of the memory upon receiving an instruction for the measurement, and
writing biological data obtained in the measurement to the memory upon completing the data erasure.
